(19) United States
(12) Patent Application Publication
(10) Pub. No.: US 2004/0121290 A1
(43) Pub. Date: Jun. 24, 2004

(54) Biocompatible implants

(75) Inventors: Zoran Minevski, The Woodlands, TX (US); Carl Nelson, Bryan, TX (US)

Correspondence Address: STREETS & STEELE, 13831 NORTHWEST FREEWAY, SUITE 355, HOUSTON, TX, 77040, US

(73) Assignees: Lynntech, Inc.; Lynntech Coatings, Ltd.

(21) Appl. No.: 10/663,626

(22) Filed: Sep. 16, 2003

Related U.S. Application Data
(63) Continuation-in-part of application No. 10/353,622, filed on Jan. 29, 2003.
(63) Continuation-in-part of application No. 10/245,821, filed on Sep. 16, 2002.
(63) Continuation-in-part of application No. 10/353,613, filed on Jan. 29, 2003.

(51) Int. Cl. A61F 2/06

(52) U.S. Cl. 433/201.1

(57) ABSTRACT

A biocompatible surgical implant or component for a surgical implant for use in human beings and animals is described. The implant has an oxide film-forming valve metal substrate, such as titanium, titanium alloy, zirconium, or zirconium alloy, or stainless steel, or cobalt-chromium-molybdenum alloy having a surface that has been treated such that phosphorous and oxygen are incorporated into the treated surface of the implant. The surface treatment carried out on the implant includes low temperature anodic treatment of the substrate in a phosphorus-containing solution, such as a phosphate-containing solution. The anodic treatment changes or modifies the substrate surface through electrochemical reactions between the substrate, acting as an anode, and phosphate ions contained in an electrolyte solution, such as provided by an aqueous solution of phosphoric acid. The phosphorus-containing solution may be substantially calcium-free. The anodic treatment is effective on various metal surfaces, including alloys containing less than 98 percent titanium.

This application is a continuation-in-part claiming priority from U.S. application Ser. No. 10/245,821 filed Sep. 16, 2002, U.S. application Ser. No. 10/353,622 filed on Jan. 29, 2003, and U.S. application Ser. No. 10/353,613 filed on Jan. 29, 2003.

### BACKGROUND OF THE INVENTION

1. Field of the Invention

The present invention relates to surgical implants, such as surgical implants used in orthopedic surgery and dentistry. More specifically, the present invention relates to surface treatments of surgical implants, the surface layers formed on the implants and a process for carrying out the surface treatments.

2. Description of the Related Art

Medical implants and prostheses provide structural and mechanical aid or replacement for parts of the body that can no longer provide their intended function. Implants are subject to stress and must bear the required loads without failure. Implants must also be corrosion resistant and biologically compatible with various body tissues, organs and fluids so that they can remain in the body for years.

Implants generally include metal wires, rods, plates, screws, tubes, and other devices. Some implants are attached to bone to reinforce damaged bone in the body. Since they are generally much stiffer than bone, implants can promote stress shielding in the attached bone leading to implant loosening and osteoporosis. Implants presently available will typically have a lifetime of about 7-10 years. While surgical implant replacement is possible, replacement surgery is usually not performed more than once for a particular implant device due to the extent of bone damage created by the first implant. As a result, recommended medical procedures involving implants are generally reserved for people over the age of 40 years. Unfortunately, many younger people injured in accidents could benefit from implants and need implants that will last for many more years than those that are currently available.

Titanium alloys are usually the materials of choice for making surgical implants. In particular, Ti-6Al-4V, a titanium alloy initially developed for aerospace applications, is currently the alloy used to make most orthopedic implants and has been described in various papers and patents. For example, U.S. Pat. No. 4,854,496 describes an implant made by diffusion bonding titanium powder to a titanium or titanium alloy, such as Ti-6Al-4V, substrate. The coating provides the implant with enhanced biocompatibility. Additional examples of coated alloy implants now follow.

U.S. Pat. No. 5,763,092 describes orthopedic and dental implants with a crystalline calcium phosphate ceramic coating known as hydroxyapatite. The coating anchors the implant to the existing bone and provides the implant with enhanced biocompatibility, thereby increasing the useful life of the implant and minimizing the likelihood of implant rejection by the body.

Orthopedic and dental implants are commonly coated with a substance to provide a surface suitable for the in-growth of bone and marrow, thereby securely anchoring the implant to the existing bone. The biocompatibility of the coating substance further minimizes implant rejection and increases the useful life of the implant. Calcium phosphate ceramics, such as tricalcium phosphate (TCP) and hydroxyapatite (HA), are particularly suitable materials. Hydroxyapatite is particularly preferred since it is a naturally occurring material in bone. However, it is difficult to satisfactorily bond hydroxyapatite to the surface of surgical implants, requiring the application of both heat and pressure. Still, the hydroxyapatite coating is subject to delamination.

Although the Ti-6Al-4V alloy is generally considered to be chemically inert, biocompatible with human tissue, and corrosion resistant to human body fluids and other corrosive environments, vanadium and aluminum are potentially toxic. Normal wear leads to implant degradation and the release of alloy elements into the body. For example, vanadium has been observed in body tissues near Ti-6Al-4V alloy implants.

A more benign replacement for titanium alloy implants may solve the problem of the release of toxic elements into the body from degraded alloy implants. An implant of pure titanium could be the ideal replacement since it is lightweight, chemically and biologically more compatible with human tissue, and can rigidly fixate to bone better than a titanium alloy implant. Unfortunately, pure titanium lacks sufficient strength for general use as an implant material. For example, Ti-6Al-4V alloy has a yield strength of about 795 MPa and an ultimate strength of 860 MPa, whereas the yield strength and ultimate strength for pure titanium are only about 380 MPa and 460 MPa, respectively.

In order to reduce the corrosion rate of implants, various coatings have been applied. For example, U.S. Pat. No. 5,211,833 discloses a method for coating implants with a dense, substantially non-porous oxide coating to minimize the release of corrosion products into the body.

U.S. Pat. No. 5,354,390 (Haszmann et al.) provides a method of forming a surgical implant with a biocompatible coating on titanium or a titanium-base microalloy containing at least 98% by weight titanium. The method involves anodic oxidation of the implant in an aqueous phosphate-containing electrolyte so that a few percentages of phosphate anions are incorporated into an oxide layer. The method includes anodic oxidation of the implant at a constant current density until reaching a target voltage between 105 V and 125 V, maintaining the target voltage for 60 minutes, then washing and heat-treating the implant at a temperature between 300° C. and 450° C. to form crystalline titanium dioxide of the anatase, brookite or rutile type before repeating the anodic oxidation and heat treating steps to develop a corrosion-resistant, coherent oxide-ceramic layer of at least 2000 Angstroms in thickness.

U.S. Pat. No. 5,478,237 (Ishizawa) provides an implant having a titanium or titanium alloy surface having an anodic oxidation film formed on the surface, wherein the film contains calcium and phosphorus. The film is formed by anodic oxidation of the implant in an electrolyte containing between 0.1 and 0.5 molar concentration of a calcium compound and between 0.07 and 0.26 molar concentration of a phosphorus compound. A final hydrothermal treatment with steam is used to form a film of a calcium phosphate compound, such as hydroxyapatite, on the anodic oxide film. While a voltage range of 10 to 600 Volts is disclosed, the voltages in the examples are limited to the range of 300 to 390 Volts.

Japanese patent application JP2-194195 discloses the formation of a thick oxide film on titanium and titanium alloys through anodic oxidation in a mixed aqueous solution containing specific amounts of both phosphoric acid and oxalic acid. Phosphoric acid concentrations less than 0.05 weight percent are said to prevent formation of a normal anodic oxide film, and concentrations greater than 2 weight percent are said to make the film macroporous. Similarly, oxalic acid concentrations less that 0.05 weight percent do not improve the quality of the anodic oxide film, and concentrations greater than 2 weight percent make the film macroporous and drops electrolytic voltage. The anodic oxidation was performed at voltages between 200 and 500 Volts.

Therefore, there is a need for strong, lightweight, corrosion resistant implants that are chemically and biologically compatible with human fluids and tissue. It would be advantageous if the biocompatibility could be provided through a surface treatment of an implant, wherein the treatment process would not require significant heat or pressure to implement and would not significantly change the overall dimensions of the implant. It would be further advantageous if bone or marrow rather than fiberous tissue would readily grow onto the surface treated implant and/or into pores on the implant and bond with the implant, rather than reject the implant as a foreign substance. Finally, it would be very advantageous if the implant provided a useful life greater than seven to ten years, so that the implant could be successfully used in younger patients.

### SUMMARY OF THE INVENTION

One embodiment of the present invention provides a biocompatible implant comprising an oxide film-forming metal substrate, such as a metal selected from titanium, titanium alloy, zirconium, zirconium alloy, and combinations thereof, wherein the substrate surface comprises oxides of the substrate metal, phosphorus atoms and oxygen atoms. In one embodiment, the phosphorus atoms are provided by a component selected from phosphorus, phosphorus oxides, titanium phosphorus oxides and combinations thereof. The phosphorus atoms may also be provided by a phosphate or phosphate-containing compound, or a neutrally charged derivative of phosphate, such as surface incorporated phosphate (PO₄) and/or phosphite (PO₃) species. Preferably, the phosphorus atoms will have a concentration between about 1 mole % and about 15 mole % at the surface of the substrate. It is also preferable to have no prior electrochemically grown layer of metal oxide(s) between the substrate and the surface comprising phosphorus and oxygen. Advantageously, the substrate may be an alloy, such as Ti-6Al-4V (ASTM grade 5 or ISO 5832-3:1996; approximately 90% Ti, 6% Al, and 4% V) or different titanium alloy that includes an element selected from molybdenum, zirconium, iron, aluminum, nickel, copper, niobium, hafnium, chromium, cobalt, manganese, ruthenium, silver, beryllium, vanadium, palladium, yttrium and combinations thereof. One example of a suitable titanium alloy is a titanium-nickel alloy having the composition of 50 atom % Ni, 50 atom % Ti (55 wt % Ni, 45 wt % Ti) sold under the tradename NITINOL (available from Johnson Matthey, Inc., San Jose, Calif.). Titanium alloys having less than 98% titanium are suitable and often preferred.

Suitably the titanium alloy may include a class of alloys which form metallic glass upon cooling below the glass transition temperature at a rate less than 10³ K/s. A preferred group of metallic glass alloys has the formula (Zrₗ₋ₓTiₓ)ₐ(Cu_{l-y}Ni_{y})_{b}Be_{c}, where x and y are atomic fractions, and a, b and c are atomic percentages. In this formula, the values of a, b and c partly depend on the proportions of zirconium and titanium. Thus, when x is in the range of from 0 to 0.15, a is in the range of from 30 to 75%, b is in the range of from 5 to 52%, and c is in the range of from 6 to 47%. When x is in the range of from 0.15 to 0.4, a is in the range of from 30 to 75%, b is in the range of from 5 to 52%, and c is in the range of from 5 to 47%. When x is in the range of from 0.4 to 0.6, a is in the range of from 35 to 75%, b is in the range of from 5 to 52%, and c is in the range of from 5 to 47%. When x is in the range of from 0.6 to 0.8, a is in the range of from 38 to 75%, b is in the range of from 5 to 52%, and c is in the range of from 5 to 42%. When x is in the range of from 0.8 to 1, a is in the range of from 38 to 75%, b is in the range of from 5 to 52%, and c is in the range of from 5 to 30%, under the constraint that 3c is up to (100-b) when b is in the range of from 10 to 43.

Furthermore, the (Zrₗ₋ₓTiₓ) moiety may also comprise additional metal selected from the group consisting of from 0 to 25% hafnium, from 0 to 20% niobium, from 0 to 15% yttrium, from 0 to 10% chromium, from 0 to 20% vanadium, from 0 to 5% molybdenum, from 0 to 5% tantalum, from 0 to 5% tungsten, and from 0 to 5% lanthanum, lanthanides, actinium and actinides. The (Cu_{l-y}Ni_{y}) moiety may also comprise additional metal selected from the group consisting of from 0 to 25% iron, from 0 to 25% cobalt, from 0 to 15% manganese and from 0 to 5% of other Group 7 to 11 metals. The beryllium moiety may also comprise additional metal selected from the group consisting of up to 15% aluminum with the beryllium content being at least 6%, up to 5% silicon and up to 5% boron. Other elements in the composition should be les than two atomic percent. Bulk metallic glass alloys of this type are disclosed in U.S. Pat. No. 5,288,344 which is incorporated herein by reference. A specific bulk metallic glass alloy has the chemical formula given by Zr_{41.2}Ti_{13.8}Ni10Cu_{12.5}Be_{22.5} and is manufactured by Liquidmetal Technologies, Tampa, Fla. and sold under the Tradename Vitreloy 1 (VIT-001 series).

Similarly, suitable bulk metallic glasses, or bulk amorphous alloys, include Zr-Al-Co-Ni-Cu alloy systems, Zr-Ti-Al-Ni-Cu alloy systems, Zr-Ti-Nb-Al-Ni-Cu alloy systems, and Zr-Ti-Hf-Al-Co-Ni-Cu alloy systems that have significantly low critical cooling rates in the range of from 1 to 100 K/s, and which are disclosed in U.S. Pat. No. 5,740,854 which is also incorporated herein by reference.

Equally suitable materials include a metal-matrix composite material having reinforcement materials bonded together by an amorphous-metal matrix. The reinforcements are most preferably refractory ceramics having melting points at least about 600° C. above the melting point of the amorphous metal matrix and also having excellent stability, strength, and hardness. Examples of these refractory ceramics useful as reinforcements include stable oxides, stable carbides, and stable nitrides.

The metal-matrix material is a bulk-solidifying amorphous material in which the amorphous state can be retained in cooling from the melt at a rate of no greater than about 500° C. per second. The metal-matrix material should have a melting point at least about 600° C., preferably more, below the melting point of the refractory material. A preferred such metal-matrix material has a composition near a eutectic composition, such as a deep eutectic composition with a eutectic temperature on the order of 660° C. One such material has a composition, in atom percent, of from about 45 to about 67 percent total of zirconium plus titanium, from about 10 to about 35 percent beryllium, and from about 10 to about 38 percent total of copper plus nickel, plus incidental impurities adding to a total of 100 atom percent. Amorphous metal/reinforcement composite materials of this type are disclosed in U.S. Pat. Nos. 5,567,251 and 5,866,254 which are also incorporated herein by reference.

Other suitable materials include a group of alloys that exhibit “super” properties, such as ultralow elastic modulus, ultrahigh strength, super elasticity, and super plasticity, at room temperature. Various alloy composition combinations exhibit these properties, such as TiₓTa₁₂Nb₉V₃Zr₆O_{y} and TiₓNb₂₃Ta_{0.7}Zr₂O_{y}. The oxygen concentration, y, is restricted in the range 0.7 to 3.0 mole % with the balance being made up by the concentration of titanium, x, in mole %.

The biocompatible implant may also comprise a substrate that includes a tantalum or tantalum alloy surface, a niobium or niobium alloy surface, a zirconium or zirconium alloy surface, a hafnium or hafnium alloy surface, a stainless steel surface, such as SS316L, or a cobalt-chromium-molybdenum alloy (ASTM F75-1987) surface. The cobalt-chromium-molybdenum alloy is characterized by superior wear resistance, hardness, and high corrosion resistance. Its chemical composition is about 25.12 wt % chromium, 5.62 wt % molybdenum, 0.77 wt % silicon, 0.72 wt % iron, and the remainder cobalt.

The implant may take many forms, but the implant specifically may be an orthopedic implant, a dental implant, an orthopedic fixation device, or a device selected from an orthopedic joint replacement and a prosthetic disc for spinal fixation. Optionally, the substrate may comprise a solid inner portion and a porous outer layer, such as sintered metal beads, fine metal mesh or the like secured to the solid inner portion. Beneficially, tissue can grow onto the surface of the solid inner portion and/or into pores in the porous outer layer. Furthermore, this tissue may be selected from, without limitation, bone, marrow and combinations thereof. It should be recognized that the porous outer layer may be made from the same material as the solid inner portion or a different material than the solid inner portion. In either case, the porous outer layer is preferably made from a biocompatible material, such as oxide film-forming titanium, titanium alloys, zirconium, zirconium alloys, other oxide film-forming metals, and combinations thereof. Optionally, the porous outer layer comprises sintered metal particles.

It is also possible for the implant to further comprise a coating of hydroxyapatite deposited on internal surfaces and external surfaces of the porous outer layer without blocking the pores. The hydroxyapatite coating may be applied by a method selected from plasma deposition, electrodeposition, and hydrothermal treatment after reacting the phosphorus-containing oxide surface with calcium.

In another embodiment, the substrate surface incorporates phosphorus to a depth (or film thickness) that may be less than about 1 micron, such as between about 0.01 microns and about 0.9 microns, and more specifically between about 0.02 microns and about 0.5 microns. Alternatively, the surface may incorporate phosphorus to a depth between about 0.02 microns and about 5 microns, or between about 0.05 microns and about 5 microns.

A specifically preferred embodiment is a biocompatible surgical implant comprising a substrate with a surface comprising phosphorus and oxygen, wherein there is no prior separate electrochemically-grown metal oxide layer between the original substrate and the layer or surface comprising phosphorus and oxygen. The substrate is preferably an oxide film-forming metal, such as a metal selected from titanium, titanium alloys, zirconium, zirconium alloys, and combinations thereof.

Further, another embodiment includes a biocompatible surgical implant, consisting at least partly of a substrate member that has been treated by anodic oxidation in the presence of phosphate.

A still further embodiment includes a surgical implant having a metal or metal alloy surface, the improvement consisting essentially of a phosphorus-containing anodic oxidation film formed on the surface. After the anodic oxidation, the surface of the phosphorus-containing oxide film is characterized in that it experiences a corrosion rate of less than 10 A/cm²×10⁻⁹ when disposed in contact with body fluids.

The present invention also provides a method comprising performing anodic phosphation on a surface of a surgical implant, wherein the surface consists substantially of a metal selected from oxide film-forming titanium, titanium alloys, zirconium, zirconium alloys, other oxide film-forming metals, or a combination thereof. Specifically, the metal alloys may contain alloying constituents such as aluminum, vanadium, nickel, copper, niobium, hafnium, chromium, cobalt, manganese, ruthenium, silver, beryllium, molybdenum, palladium, yttrium, and zirconium. The surgical implant formed by this method is also expressly included within the scope of the present invention. In one embodiment, the step of performing anodic phosphation further comprises disposing the surface into a solution containing phosphate ions, and applying an anodic electrical potential to the surface. This method is characterized in that the surface is modified to comprise phosphorus and oxygen. The solution may include, without limitation, an electrolyte solution or an aqueous solution, such as an aqueous solution comprising greater than 10% water by volume or an aqueous solution of phosphoric acid. Preferably, the solution is substantially free from alcohol. A preferred solution is an aqueous phosphoric acid solution having a phosphoric acid concentration of between about 0.01 N and 5.0 N, most preferably between about 0.1 N and about 3.0 N. The temperature of the solution is preferably between about 15° C. and about 65° C. during the application of electrical potential, or electrical current, and more preferably between about 25° C. and about 55° C. during the application of electrical potential, or electrical current. Alternatively, the temperature of the solution is at least 25° C. during the application of electrical potential, or electrical current. The anodic phosphation will preferably be performed on a surface that has no previous electrochemically grown layer of titanium oxide. The electrical potential may be, without limitation, between about 10 volts and about 150 volts, or between about 25 volts and about 100 volts. Alternatively, the electrical potential may be greater than 25 volts. It will be recognized by one skilled in the art of anodization of metals that anodic oxidation of surgical implants in the presence of phosphates may be carried out in a divided or undivided electrochemical cell. In a divided electrochemical cell a counter electrode, or cathode, is placed in a first compartment and the surgical implant work piece is placed in a second compartment. A reference electrode, such as a silver/silver chloride electrode or a standard hydrogen electrode, in a third compartment may also be used. It will also be recognized by one skilled in the art of anodization of metals that anodic oxidation of surgical implants may be carried out by: (i) applying a controlled electrical potential between the surgical implant work piece and a counter electrode, or a cathode, for a period of time; (ii) applying a controlled electrical current between the surgical implant work piece and a counter electrode, or cathode, for a period of time; or (iii) applying a controlled electrode potential between the surgical implant work piece and a reference electrode while allowing current to flow between the surgical implant work piece and a counter electrode, or cathode, for a period of time. Further, it will be recognized by one skilled in the art of anodization of metals that the anodic phosphation of surgical implants is preferably carried out by: (i) applying a controlled direct current (DC) electrical potential; (ii) applying a controlled direct current (DC) electrical current; or (iii) applying a controlled direct current electrode potential between the surgical implant work piece and a reference electrode. Examples of controlled electrical potential, controlled electrical current, or controlled electrode potential comprise, constant, linear ramp at various ramp rates, staircase at various ramp rates, square wave, or other geometric wave forms of electrical potential, electrical current or electrode potential. Specifically, it is preferred that the implant be subjected to the controlled electrical potential, or controlled electrical current as the case maybe, for between about 15 seconds and about 2 hours, more specifically between about 1 minute and about 30 minutes.

In another embodiment, the method may further comprise disposing the implant substrate in a detergent before disposing the implant substrate in the solution. In a still further embodiment, the method may further comprise removing passive oxide films from the surface of the implant substrate before performing anodic oxidation, such as by disposing the substrate in a fluoroboric acid solution. The method further comprises applying a cathodic potential, or cathodic current, to a cathode, or counter electrode in the solution, wherein the cathode material, or counter electrode, is preferably selected from platinum, palladium, graphite, gold, titanium, platinized titanium, palladized titanium, and combinations thereof.

Yet another embodiment provides a method comprising performing anodic oxidation on a metal surface of a surgical implant in the presence of phosphates, wherein the surface has no electrochemically grown layer of metal oxide(s) prior to anodic oxidation in an aqueous solution containing phosphates. The invention specifically includes the surgical implant formed by this method.

Still further, there is provided a method for surface modification of metallic portions of a surgical implant, comprising performing anodic oxidation with phosphates on a surgical implant having no prior electrochemically grown layer of metal oxide(s). Preferably, the surgical implant is made of one or more of the metal substrate materials set out above.

Additionally, an embodiment provides a method of preparing a biocompatible surgical implant, consisting essentially of performing anodic oxidation on a metal or metal alloy surgical implant in the presence of phosphates. Again, the metal or metal alloys are preferably selected from passive oxide film-forming titanium, titanium alloys, zirconium, zirconium alloys, other oxide film-forming metals, and combinations thereof. An additional method comprises implanting a device into an animal or human, wherein the device comprises a metal or metal alloy substrate covered with a film comprising phosphorus and oxides of the metal or metal alloy. Preferably, the substrate or substrate surface comprises Ti-6Al-4V prior to anodic phosphation. Alternatively, the metal may be an alloy that includes an element selected from molybdenum, zirconium, iron, aluminum, nickel, copper, niobium, hafnium, chromium, cobalt, manganese, ruthenium, silver, beryllium, palladium, vanadium and combinations thereof. The implant device may be, without limitation, an orthopedic implant or a dental implant. Preferably, the external surface is porous, such as wherein tissue of the human or animal can grow into pores of the porous surface. Such tissue includes, without limitation, tissue selected from bone, marrow and combinations thereof. Optionally, the porous external surface comprises sintered metal particles or sintered metal mesh. As stated in other embodiments, the surface comprises phosphorus and oxygen. The depth of the phosphorus and/or oxygen penetration may vary, such as no more than about 1 micron, between about 0.01 microns and about 0.9 microns, between about 0.02 microns and about 0.5 microns, between about 0.05 microns and about 0.5 microns, or greater than about 1 micron.

The foregoing and other objects, features and advantages of the invention will be apparent from the following more particular description of a preferred embodiment of the invention, as illustrated in the accompanying drawing wherein like reference numbers represent like parts of the invention.

### BRIEF DESCRIPTION OF THE DRAWINGS

FIG. 1 is a cross sectional view of an orthopedic surgical implant in accordance with the present invention.

### DETAILED DESCRIPTION

The present invention provides an apparatus that may be used as a biocompatible implant in human beings and animals. The present invention further provides a method for making a biocompatible implant. The implants may take many different shapes and forms, such as screws, wires, rods, plates, and tubes, but all the implants of the present invention have a substrate surface that has been electrochemically modified to comprise phosphorus atoms (perhaps in the form of phosphate) and oxygen atoms (perhaps in the form of phosphates and metal oxides), as well as the metal or metal alloy of the substrate (perhaps in the form of metal oxides). The implant substrate is a material selected from passive oxide film-forming metals, stainless steel, a cobalt-chromium-molybdenum alloy, and combinations thereof. Passive oxide film-forming metals include, without limitation, titanium, titanium alloys, zirconium, and zirconium alloys. Specific examples of these substrates include a titanium-nickel alloy (such as Nitinol), a cobalt-chromium-molybdenum alloy (such as ASTM F75-1987), SS316, and Ti-6Al-4V. Accordingly, it is not necessary to provide a coating or layer that physically covers the entire surface of the implant substrate.

The surface treatment that is performed on the implant substrate includes anodic oxidation in the presence of phosphates or other suitable phosphorus-containing species. Anodic oxidation does not deposit on, or coat, the surface of the implant with a deposit or coating, but rather converts or modifies the substrate surface through electrochemical reactions between the substrate, acting as an anode, and phosphate ions and water molecules. The phosphates ions and water molecules are contained in an electrolyte solution, such as provided by an aqueous solution of phosphoric acid. One advantage of this surface treatment over a coating is that the dimensions of the implant do not significantly change. This is important because the surface modification process allows the surgical implant substrate to be constructed to exact dimensions without having to account for the thickness of additional coatings being applied to the implant. Another advantage is that the electrochemically grown anodic oxidation film has a stronger adhesion strength with the substrate than does a distinct coating (such as, plasma sprayed hydroxyapatite).

The surface treatment incorporates phosphorus atoms and oxygen atoms into an anodic oxidation film formed on the substrate. Without being limited to any particular theory of the composition at the substrate surface, it is believed that the surface treatment incorporates phosphate-like species and/or derivatives of phosphate into a portion of the substrate and may additionally convert some of the metal atoms at the surface of the substrate to corresponding metal oxides. It may be that the phosphate-like species, and/or derivatives of phosphate, are incorporated into the anodically grown titanium oxide and/or other metal oxides. Regardless of the fact that the exact composition or structure of the modified surface is not known with certainty, the concentration of the phosphorus-containing species, such as phosphate, derivatives of phosphate such as phosphite, and/or metal phosphorus oxides, at the surface of the substrate is preferably between about 1 mole % and about 15 mole %. The surface treatment preferably incorporates phosphorus-containing species into the substrate or film to a depth or thickness of between about 0.02 μm and about 0.5 μm. Thicker layers are possible, specifically including up to about 5 μm. As used here, the terms “phosphorus atoms” and “oxygen atoms” are meant to encompass elemental, ionic and molecular forms.

Furthermore, the phosphate-containing, aqueous electrolyte solution is preferably provided without any appreciable amount of any calcium compound, and is most preferably calcium-free.

It is also possible for the implant to further comprise a coating of hydroxyapatite deposited on internal surfaces and external surfaces of the porous outer layer without blocking the pores. The hydroxyapatite coating may be applied by a method selected from plasma deposition, electrodeposition, and hydrothermal treatment after reacting the phosphorus-containing oxide surface with calcium. However, it is believed that the formation of calcium-phosphate or hydroxyapatite over the treated surface of the implant may be less desirable than using the implant with the anodically produced phosphorus-containing oxide surface. Whereas hydroxyapatite is known to be biocompatible, the anodically produced phosphorus-containing oxide surface is bioenhancing or biostimulating as well as being biocompatible. This revelation was not expected, but is supported by the data from the examples below. The most significant advantage provided by the surface treatments of the present invention is the unique biocompatibility and bioenhancement that is provided by the anodically produced phosphorus-containing metal oxide surface. The biocompatible implant of the present invention provides a surface that is suitable for the predominant in-growth of desirable bone and marrow tissues and the suppression of undesirable fibrous (scar) tissue, thereby helping to securely anchor the surgical or dental implant to existing bone. A porous layer may be provided to the implant initially to host new tissue growth by covering at least a portion of the surface of the implant with sintered metal spheres, sintered metal wire mesh or the like of a substrate metal. Rejection of the implant by the body is minimized and the useful life of the implant is increased because the implant is surrounded with a predominant amount of in-grown bone and marrow tissues. The porous outer layer bonded to the solid inner portion of the implant may be of the same material as the solid inner portion or it may be of a different material. Another significant advantage provided by the surface treatments of the present invention is the unique bioenhancement that is provided by the anodically produced phosphorous-containing metal oxide surface of the solid inner portion of the implant. Unexpected growth of a predominance of desirable bone and marrow tissues occur at this modified surface of the solid inner portion of the implant. This may avoid the need for a porous outer layer being applied to a surgical implant.

Perhaps the most significant advantage provided by the surface treatments of the present invention is the biocompatibility or bioenhancement that is provided by the phosphorus-containing metal oxide surface. The biocompatible implant of the present invention provides a surface that is suitable for in-growth of bone tissue, thereby helping to securely anchor the surgical or dental implant to existing bone. A porous layer may be provided to the implant initially to host new tissue growth by covering at least a portion of the surface of the implant with sintered spheres, sintered wire mesh or the like of a substrate metal. Rejection of the implant by the body is minimized and the useful life of the implant is increased because the implant is surrounded with in-grown tissue. The porous outer layer bonded to the solid inner portion of the implant may be of the same material as the solid inner portion or it may be of a different material.

Another important advantage of the present surface treatments is the increased corrosion resistance that the treatment provides to the substrate, without forming extremely thick layers. There is concern for the toxicological effects of corrosion products that can be released from existing implants into the body and contaminate adjoining tissue. In general, metal toxicity can result in metabolic alterations, alterations in host/parasite interactions, immunological interactions, non-specific immunological suppression due to the antichemotactic properties, and chemical carcinogenesis. The surface treatment of the present invention provides excellent corrosion protection of metal implants and minimizes toxicological effects.

The greater the phosphorus concentration (phosphate-like species and/or derivatives of phosphate) present in the surface of the implant substrate, the greater is both the resistance to corrosion and the biocompatibility. The phosphorus concentration may be controlled during the electrolytic surface treatment using controlled voltage (or controlled current), electrolysis time, temperature and concentration of the phosphate source, such as H₃PO₄, that is used as the electrolyte. By controlling these parameters, the concentration of phosphorus atoms in the surface of the implant may vary from less than 1.5 mole % to greater than 8.5 mole %. Table 1 illustrates how the corrosion resistance increased (lower corrosion rate, more positive corrosion potential, and higher polarization resistance) of a Ti-6Al-4V alloy as cell voltage (potential), time, temperature and concentration of the phosphoric acid are varied during the electrolysis procedure.

TABLE I
Summary of Corrosion Resistance Data of Surface Treated
and Untreated Ti—6Al—4V Alloy Substrates

| | | CORROSION | CORROSION | POLARIZATION |
|---|---|---|---|---|
| | | RATE | POTENTIAL | RESISTANCE |
| PARAMETERS FOR PHOSPHATION | | (A/cm² × 10⁻⁹) | (V) | (ohms/cm² × 10⁵) |
| Ti—6Al—4V WITHOUT PHOSPHATE LAYER | | 88 | −0.353 | 1.94 |
| POTENTIAL, E (V) | 25 | 6.5 | −0.082 | 2.53 |
| t = 3 min; T = 25° C.; C = 1.0 N | 50 | 4.9 | −0.037 | 4.78 |
| | 75 | 3.4 | +0.098 | 7.66 |
| | 100 | 1.9 | +0.290 | 10.9 |
| TIME, t (min) | 1 | 7.7 | −0.105 | 2.32 |
| E = 25 V; T = 25° C.; C = 1.0 N | 10 | 6.2 | −0.040 | 3.23 |
| | 30 | 4.4 | +0.015 | 5.26 |
| TEMPERATURE, T (° C.) | 35 | 4.8 | 0.047 | 4.89 |
| E = 25 V; t = 3 min; C = 1.0 N | 45 | 3.1 | 0.103 | 8.23 |
| CONCENTRATION OF H₃PO₄, c (N) | 0.1 | 21 | −0.197 | 2.07 |
| E = 25 V; t = 3 min; T = 25° C. | 0.5 | 9.2 | −0.135 | 2.37 |
| | 3.0 | 4.1 | +0.075 | 6.47 |

Corrosion rates were measured in a solution that simulated body fluids (blood and tissue). Ethylenediaminetetraacetate, EDTA, was chosen as a complexing agent to model or simulate the effects of proteins and biomolecules on the solution kinetics. Solution kinetics were studied in 8 mM EDTA with a simulated interstitial electrolyte consisting of various salts, NaCl, CaSO₄, CaCl₂, and glucose. 4.5 mM glucose was added to simulate its normal concentration in blood. This solution simulates the activity of serum with the use of EDTA as the chelating agent for the metal ions released from the metal surface of the substrate in vivo so that these ions do not remain in solution around the metal surface. Rather, the metal ions form complex molecules that are transported away from the metal surface through motion of the fluid. As a result, steady state equilibrium of the dissolution and reprecipitation is never achieved. The rates of corrosion in this simulated environment are shown in Table 1.

It is seen that the control coupon (non-treated Ti-6Al-4V) exhibits a much more negative open-circuit potential than all the other electrodes having a phosphorus-containing metal oxide film, indicating that untreated samples are more likely to corrode than those that are oxidized in accordance with the present invention.

The impedance responses obtained for the treated Ti-6Al-4V alloy surfaces were similar in shape but different in size as shown in Table 1. This indicates that the same fundamental process occurred on all the specimens, with a different corrosion protection in each case. Since the resistive contribution is directly proportional to corrosion protection (e.g. higher resistance gives higher corrosion protection), it is evident from Table 1 that phosphate-enhanced anodic oxidation of metal substrate surfaces yielded greater corrosion resistance with much higher values of polarization resistance (Rct). In addition, corrosion rates corresponding to high polarization resistance of the phosphate-containing or phosphorus-containing metal oxide surfaces are smaller than that of the specimens that were not treated by a factor of six. These studies show that the phosphated metal surfaces in contact with EDTA/SIE are corrosion resistant and that this corrosion resistance is directly proportional to the phosphorus concentration in the metal surface.

The wear behavior of a control titanium alloy sample as well as titanium alloy samples phosphated at 25, 75, and 100 V were performed using a pin-on-disk test rig. Flat Ti-6Al-4V disks were mechanically ground with diamond paste, followed by a silicon polishing solution. A mirror quality finish with an average surface roughness (Rₐ) less than 0.03 μm was obtained. Titanium alloy disks and pins made of ultra-high molecular weight polyethylene (UHMWPE, contact area 1.5 mm²) and physiological solution (EDTA/SIE) as lubricant were used in wear testing. Constant normal force (F_{N}) of 15 N was applied, resulting in a pressure of 10 MPa. A sliding velocity of 5 cm/s and test durations of up to 36 hours were chosen. To determine the coefficient of friction, μ (μ=F_{R}/F_{M}), the friction force, F_{R}, was recorded during the experiments. Volumetric UHMWPE wear was determined by measuring the decrease in the length of the pins using a digital caliper (resolution of 0.01 mm). The sliding surfaces and the wear particles were investigated using light microscopy. Although pin-on-disk experiments do not replicate the tribological conditions in vivo (with respect to type of motion or dynamic loading), they have been known to be used as cleaning tests.

The untreated control coupon showed severe wear with rupturing of the titanium alloy surface and abrasion of black particles after only a few revolutions. While the sample treated at 25 V showed moderate abrasion, samples treated at 75 and 100 V showed smooth features after 5×10⁴ revolutions.

Titanium may be alloyed with several different elements to provide a preferred alloy for implants. These elements may be, for example, molybdenum, zirconium, iron, aluminum, nickel, copper, niobium, hafnium, chromium, cobalt, manganese, ruthenium, silver, beryllium, vanadium, palladium, yttrium and combinations thereof. Zirconium may also be alloyed with several different elements, including, for example, molybdenum, iron, aluminum, vanadium, titanium, palladium, yttrium and combinations thereof.

The implants may be of any type, such as orthopedic implants or dental implants. Specifically, the orthopedic implants may include, without limitation, a fixation device, an orthopedic joint replacement or a prosthetic disc for spinal fixation.

On completing anodic oxidation of certain portions of the surface of a surgical implant in a phosphate-containing solution, it may be advantageous to subject those portions of the surface of the surgical implant that were not anodically oxidized to a chemical passivation treatment. Passivation is advantageously carried out by immersing those portions of the surface of the surgical implant to be passivated in 20 to 45 volume percent nitric acid (specific gravity 1.1197 to 1.285) at room temperature for a minimum of thirty minutes. If acceleration of the process is desired, a 20 to 25 percent acid solution heated to 40° C. to 60° C., may be used for a minimum of 20 minutes. For surgical implant designs in which liquid acid could be trapped, a neutralizing step involving a dilute aqueous solution of an alkali hydroxide should be used subsequent to the acid passivation step. The surgical implant should be thoroughly rinsed with deionized water after the acid passivation step, and after the neutralization step, if used, and then dried. The passivated surgical implant can be dried in air at temperatures in the range 20° C. to 70° C., preferably from 25° C. to 45° C. After completing the passivation treatment, the surgical implant can be packaged.

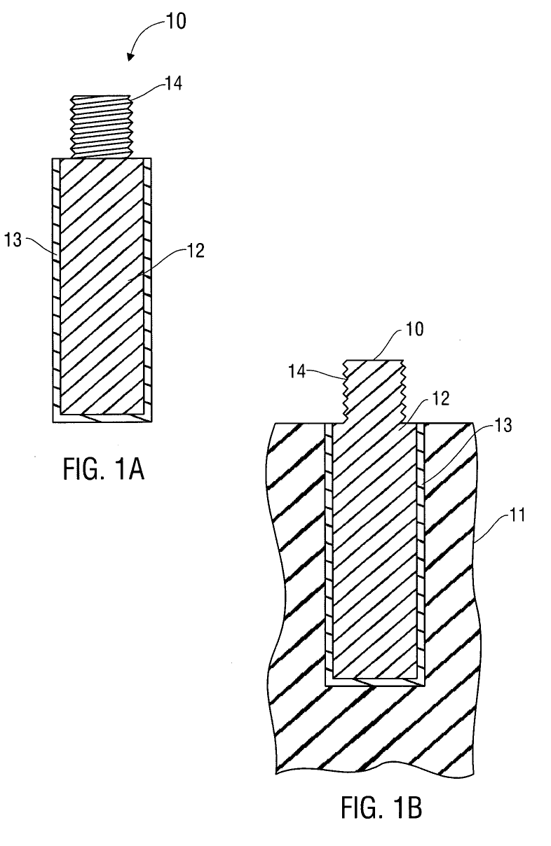

FIG. 1A is a side view of an orthopedic surgical implant 10 in accordance with the present invention and FIG. 1B is a cross-sectional view of the same orthopedic surgical implant 10 shown imbedded in the end of a bone 11. The implant 10 comprises an inner portion 12 surrounded by a porous layer 13 that is bonded to the inner portion 12 that is typically a solid or has very little porosity. The porous layer 13 shown here may be made of small diameter metal spheres that have been sintered together to form a very porous layer or shell, or a sintered fine metal mesh or the like 13. An optional threaded connection 14 is shown at one end for coupling the implant 10 with other implant devices, such as an artificial joint.

The surface modification method of the present invention is performed on a surgical implant made of material selected from oxide film-forming metals, such as titanium, titanium alloys, zirconium, zirconium alloys, and combinations thereof. In accordance with an optional but preferred pretreatment before the surface modification, the implant is first submerged in an aqueous industrial detergent with light sonication to remove oil and dirt from the surface. After rinsing with deionized water, the implant is bead blasted or otherwise treated (etched, polished, or buffed) to remove unwanted inorganic-based or organic-based surface layers or films to prepare for the surface treatment. Roughening the metal surface facilitates the accumulation of phosphate-like species at the implant surface during the surface treatment. The final step of the pretreatment is to immerse the implant into a 10% solution of HBF₄ for about one minute to remove any passive oxide film from the surface of the implant. Any acid, but preferably an acid having a fluorine-containing anion, may be used to remove the passive oxide film so long as the acid does not damage the implant.

After washing any remaining acid from the implant, the implant is submerged as the anode in the electrolyte of an electrolytic cell. The electrolyte may be any phosphate ion-containing solution, but aqueous H₃PO₄ is the preferred electrolyte. The cathode may be made of any material, preferably selected from platinum, palladium, gold, titanium, graphite, platinized titanium, and palladized titanium, but platinized titanium is the most preferred cathode material. A DC voltage is then applied across the electrolytic cell for the required period of time to provide the surface treatment or modification.

The amount of phosphate-like species incorporated in the surface or film of the implant at the end of the surface treatment is dependent upon process conditions, such as the concentration of phosphate ions in the electrolyte, the time that the implant spent in the electrolytic cell, the temperature of the cell, and the applied controlled voltage (or controlled current) across the cell. The phosphate ion concentration in the electrolyte solution may be as high as available, such as an 85 weight percent phosphoric acid, but it is preferably between about 0.01 N and about 3.5 N. More preferably, the concentration of phosphate ions in the electrolyte is between about 0.1 N and about 3 N. The temperature of the electrolyte is preferably maintained at a temperature between about 15° C. and about 65° C., most preferably between about 25° C. and about 55° C. The applied cell voltage is preferably maintained between about 10 V and about 150 V, most preferably between about 25 V and about 100 V. The surface treatment is preferably performed over a time period of between about 15 seconds and about 2 hours, most preferably between about 1 minute and about 30 minutes.

A suitable phosphate solution may include phosphoric acid, primary orthophosphates (ammonium, potassium, or sodium forms), secondary orthophosphates, tertiary orthophosphates, methyl ethyl phosphate, glycerophosphates, 1-hydroxyethane-1,1-bis-phosphonate and phytic acid. More specifically, a phosphate solution may include sodium phosphate, disodium hydrogen phosphate, sodium dihydrogen phosphate, potassium phosphate, dipotassium hydrogen phosphate, potassium dihydrogen phosphate. Combinations of the foregoing phosphates may also be used.

The substrates used in the following examples were treated to remove any existing metal oxide films on the surface thereof. The anodic oxidation was carried out without calcium in the electrolyte solutions and the anodic oxidation films had no subsequent contact with any source of calcium. In addition, the anodic oxidation film did not undergo any hydrothermal treatment or other process to generate or deposit calcium-phosphate or hydroxyapatite. Still, the anodic oxidation process was not repeated.

### EXAMPLE 1

A titanium alloy coupon made of the alloy Ti-6Al-4V and measuring 3.81 cm×5.08 cm×0.2 cm was immersed in an aqueous industrial detergent and sonicated for about 30 minutes to remove surface oil and dirt. After rinsing with deionized water, the coupon was bead-blasted at about 40 to 60 psi to roughen the coupon. After again rinsing with deionized water, the coupon was then immersed in a 10% solution of HBF₄ for about 1 minute, to remove the passive oxide film.

After again washing with deionized water, the coupon was placed in an electrolytic cell as the anode. The electrolyte in the cell was an aqueous solution of 1.0 N H₃PO4 (phosphoric acid), the applied voltage was 50 volts, and the voltage was applied for 3 minutes at an electrolyte temperature of 25° C. The coupon was then removed from the cell and exhibited a strong gold color on the surface. The coupon was rinsed with deionized water to remove traces of the mineral acid.

### EXAMPLE 2

Using the same size Ti-6Al-4V coupon and pretreatment steps as in Example 1, a coupon was placed in an electrolytic cell as the anode. The electrolyte in the cell was an aqueous solution of 1.0 N H₃PO₄, the applied cell voltage was 75 volts, and the voltage was applied for 3 minutes at an electrolyte temperature of 25° C. The coupon was then removed from the cell bearing a strong purple color on the surface. The coupon was rinsed with deionized water to remove traces of the mineral acid.

### EXAMPLE 3

A cylindrical coupon of Ti-6Al-4V measuring 3.81 cm in diameter and 0.15 cm in thickness was immersed in an aqueous industrial detergent and sonicated for 30 minutes. The coupon was polished with a diamond paste to a mirror finish and then immersed in a 10% HBF₄ solution for about 1 minute to remove the passive oxide film. After washing with deionized water, the coupon was placed in an electrolytic cell as the anode. The electrolyte in the cell was an aqueous solution of 1.0 N H₃PO₄, the applied voltage was 25 volts, and the voltage was applied for 3 minutes at an electrolyte temperature of 25° C. The coupon was then removed from the cell bearing a strong blue color on the surface. The coupon was rinsed with deionized water to remove traces of the mineral acid.

### EXAMPLE 4

Seven implants having a Ti-6Al-4V alloy core covered with a porous titanium layer bonded to the alloy surface were pretreated as in Example 1. The implants were hip replacement prostheses custom made by Wright Medical Technology of Arlington, Tenn. Each implant was placed in an electrolytic cell as the anode. The electrolyte in the cell was an aqueous solution of 0.33 N H₃PO₄, the applied voltage was 50 volts, and the voltage was applied for 30 minutes at an electrolyte temperature of 25° C. The implants emerged from the cells having the same strong gold color as the coupon in Example 1.

The treated implants were inserted into the proximal humerus of seven dogs. An additional seven implants, which were not treated, were inserted in seven other dogs as a control group. After 6 months, the amount of various tissues directly attached to the treated surfaces of the Ti-6Al-4V alloy implants and within the porous layer was quantified from histological sections. As may be seen from Table 2, the implants having the phosphate surface treatment had significantly more bone and marrow tissue and less fibrous tissue directly attached to the treated surfaces of the Ti-6Al-4V alloys than the control implant group.

TABLE 2
Percent Tissue Directly Attached to the Phosphated
and Non-Phosphated Ti-6A1-4V Alloy Substrates

| | | | | |
|---|---|---|---|---|
| Sample | | | | Metal |
| No. | Bone | Marrow | Fibrous | Beads |

Electrolytic Phosphate Surface Treated Implants

| | | | | |
|---|---|---|---|---|
| 1 | 26.2 | 18.0 | 35.8 | 19.9 |
| 2 | 24.4 | 19.0 | 31.9 | 24.6 |
| 3 | 18.5 | 18.0 | 41.7 | 21.8 |
| 4 | 52.3 | 21.7 | 4.6 | 21.4 |
| 5 | 17.6 | 13.4 | 42.8 | 26.2 |
| 6 | 44.2 | 13.8 | 22.0 | 20.1 |
| 7 | 12.9 | 4.5 | 62.5 | 20.1 |
| MEAN | 28.0 | 15.5 | 34.5 | 22.0 |

Untreated Control Implants

| | | | | |
|---|---|---|---|---|
| 1 | 0.0 | 0.0 | 84.6 | 15.4 |
| 2 | 4.2 | 3.3 | 71.1 | 21.4 |
| 3 | 25.3 | 9.9 | 44.5 | 20.3 |
| 4 | 9.4 | 3.9 | 64.2 | 22.6 |
| 5 | 12.1 | 16.2 | 45.2 | 26.6 |
| 6 | 17.8 | 3.8 | 58.9 | 19.5 |
| 7 | 9.2 | 2.4 | 64.9 | 23.6 |
| MEAN | 11.1 | 5.6 | 61.9 | 21.3 |

### EXAMPLE 5

Coupons of Ti-6Al-4V titanium alloy, measuring 50 mm×10 mm×2 mm were surface treated using the method described in Example 1. Each of the samples was exposed to varying conditions of electrolyte temperature, cell voltage, anodic phosphation processing time and phosphoric acid concentration during the electrolysis as shown in Table 3. Hydroxyapatite was then deposited on each of the surface-modified coupons, as well as non-surface-treated coupons, using plasma deposition.

The plasma deposition method included using an atmospheric plasma spraying technique. Argon was used as the carrier gas with the plasma reaching temperatures near 5000° C. The coupon was kept at a temperature under 300° C. to preserve the original mechanical properties of the metal substrate, including the modified surface. A α-β acicular microstructure was produced, presenting a yield strength of 865 MPa and an elongation of 16%.

Adhesion and tensile tests were performed on the control coupons and phosphated Ti-6Al-4V coupons according to a modification of ASTM C 633 test, which includes coating one face of a substrate fixture, bonding this coating to the face of a loading fixture, and subjecting this assembly of coating and fixtures to a tensile load normal to the plane of the coating. Each sample was glued to an upper roughened titanium grid by a special adhesive bonding glue (METCO EP15), which is a commercial high viscosity dental bonding agent.

As may be seen from the results shown in Table 3, the value of the tensile strength increased with the increase of the phosphorous concentration in the modified surface of the titanium alloy sample. Furthermore, the anodic phosphation surface treatment tended to improve the bonding strength between the coupon and the hydroxyapatite coating by a factor of 2 when compared with the non-phosphated coupons.

TABLE 3
Tensile Strength of Hydroxyapatite-Coated Anodically
Phosphated Ti—6Al—4V Alloy Substrates

| | | | Tensile |
|---|---|---|---|
| PARAMETERS FOR ANODIC | | | Strength (MPa) |
| PHOSPHATION | | | Plasma Deposited |
| SURFACE TREATMENT | | | Hydroxyapatite |
| Potential (E(V)) | 25 | V | 13.24 |
| t = 3 min; T = 25° C.; C = 1.0 N | 50 | V | 18.36 |
| | 75 | V | 20.75 |
| | 100 | V | 23.51 |
| Time (t (min)) | 1 | min | 11.47 |
| E = 25 V; T = 25° C.; C = 1.0 N | 10 | min | 15.56 |
| | 30 | min | 18.87 |
| Temperature (T(° C.)) | 35° | C. | 17.92 |
| E = 25 V; t = 3; C = 1.0 N | 45° | C. | 21.17 |
| Concentration of P₃O₄ (C(N)) | 0.1 | N | 10.92 |
| E = 25 V; t = 3 min; T = 25° C. | 0.5 | N | 12.21 |
| | 3.0 | N | 20.56 |
| Control - No Phosphorous-containing | | | 10.32 |
| Layer | | | |

The terms “comprising,” “including,” and “having,” as used in the claims and specification herein, shall be considered as indicating an open group that may include other elements not specified. The term “consisting essentially of,” as used in the claims and specification herein, shall be considered as indicating a partially open group that may include other elements not specified, so long as those other elements do not materially alter the basic and novel characteristics of the claimed invention. The terms “a,” “an,” and the singular forms of words shall be taken to include the plural form of the same words, such that the terms mean that one or more of something is provided. For example, the phrase “a solution comprising a phosphorus-containing compound” should be read to describe a solution having one or more phosporus-containing compound. The term “one” or “single” shall be used to indicate that one and only one of something is intended. Similarly, other specific integer values, such as “two,” are used when a specific number of things is intended. The terms “preferably,” “preferred,” “prefer,” “optionally,” “may,” and similar terms are used to indicate that an item, condition or step being referred to is an optional (not required) feature of the invention.

It should be understood from the foregoing description that various modifications and changes may be made in the preferred embodiments of the present invention without departing from its true spirit. It is intended that this foregoing description is for purposes of illustration only and should not be construed in a limiting sense. Only the language of the following claims should limit the scope of this invention.

What is claimed is:

1. A method for improving the biocompatibility of a surgical implant or component for a surgical implant, comprising:
anodically treating at least a portion of a surface of the surgical implant or component that is disposed in a substantially calcium-free solution of a phosphorus-containing compound, wherein the surface to be treated comprises a metal selected from the group consisting of titanium, molybdenum, zirconium, nickel, copper, iron, aluminum, vanadium, chromium, cobalt, manganese, ruthenium, silver, beryllium, palladium, yttrium, tantalum, niobium, hafnium, and combinations thereof, and wherein the titanium content of the metal is less than 98 percent titanium.

2. The method of claim 1, wherein the metal is selected from the group consisting of zirconium alloy, stainless steel alloy, titanium alloy having less than 98 percent titanium, and combinations thereof.

3. The method of claims 1, wherein the phosphorus-containing compound is selected from the group consisting of phosphoric acid, alkali metal dihydrogen phosphate, alkali metal hydrogen phosphate, and combinations thereof.

4. The method of any of claims 1, wherein the solution is an electrolyte solution.

5. The method of any of claims 1, wherein the solution is an aqueous solution comprising greater than 10% water by volume.

6. The method of any of claims 1, wherein the solution is an aqueous solution of phosphoric acid.

7. The method of claim 6, wherein the concentration of the aqueous phosphoric acid solution is between about 0.01 N and 5.0 N.

8. The method of claim 6, wherein the concentration of the aqueous phosphoric acid solution is between about 0.1 N and about 3.0 N.

9. The method of any of claims 1, wherein the substantially calcium-free solution has less than a 0.04 Molar concentration of a calcium compound.

10. The method of any of claims 1, wherein the substantially calcium-free solution has a sufficiently low calcium concentration to avoid forming a calcium phosphate coating.

11. The method of claim 1, wherein the solution is substantially free from alcohol.

12. The method of claim 1, wherein the solution has a temperature between about 15° C. and about 65° C. during the application of electrical potential.

13. The method of claim 12, wherein the temperature of the solution is between about 25° C. and about 55° C. during the application of electrical potential.

14. The method of claim 1, wherein the solution has a temperature of at least 25° C. during the application of electrical potential.

15. The method of claim 1, wherein the electrical potential is controlled between about 10 volts and about 150 volts.

16. The method of claim 15, wherein the electrical potential is controlled between about 25 volts and about 100 volts.

17. The method of claim 1, wherein the anodic treatment is performed at a controlled electrical potential greater than 25 volts.

18. The method of claim 1, wherein the implant is anodically treated under an electrical potential for between about 15 seconds and about 1 hour.

19. The method of claim 18, wherein the implant is subjected to the electrical potential for between about 1 minute and about 30 minutes.

20. The method of claim 1, further comprising:
disposing the implant in a detergent before disposing the implant in the solution.

21. The method of claim 1, further comprising:
removing oxide films from the surface of the implant before performing the anodic treatment.

22. The method of claim 21, wherein the oxide films are removed by disposing the implant in an acid solution.

23. The method of claim 1, further comprising:
applying cathodic potential to a cathode in the solution, wherein the cathode material is selected from platinum, palladium, graphite, gold, titanium, platinized titanium, palladized titanium, and combinations thereof.

24. The method of claim 1, wherein the surface has no electrochemically grown layer of titanium oxide prior to anodic oxidation.

25. The method of according to claim 1, wherein the surface is formed at least partly of a titanium alloy which includes an element selected from molybdenum, zirconium, iron, aluminum, vanadium and combinations thereof.

26. The method of claim 25, wherein the titanium alloy is Ti-6Al-4V.

27. The method of claim 1, wherein an anodic treatment forms a film having a thickness less than 2000 Angstroms.

28. A surgical implant formed by the method of claim 1.

29. The method of claim 1, wherein the surgical implant is an orthopedic implant.

30. The method of claim 1, wherein the surgical implant is a dental implant.

31. The method of claim 29, wherein the external surface is porous.

32. The method of claim 31, wherein the porous surface is such that tissue of the human or animal can grow into pores of the porous surface.

33. The method of claim 32, wherein the tissue is selected from bone, marrow and combinations thereof.

34. The method of claim 32, wherein the porous external surface comprises sintered metal particles.

35. The method of claim 31, wherein the surface comprises phosphorus and oxygen to a depth of no more than about 1 micron.

36. The method of claim 35, wherein the surface comprises phosphorus and oxygen to a depth between about 0.1 microns and about 0.9 microns.

37. The method of claim 36, wherein the surface comprises phosphorus and oxygen to a depth between about 0.2 microns and about 0.5 microns.

38. The method of claim 1, wherein the surface comprises phosphorus and oxygen to a depth between about 0.1 microns and about 5 microns.

39. The method of claim 1, wherein the surface comprises phosphorus and oxygen to a depth greater than about 1 micron.

40. The method of claim 1, further comprising:
depositing hydroxyapatite over the anodically treated surface, wherein the hydroxyapatite is applied by a method selected from plasma deposition and electrodeposition.

41. A method, comprising:
anodically treating a first portion of a metal surgical implant or metal component for a surgical implant in a solution comprising a phosphorus-containing compound; and
passivating a second portion of the metal in a different solution.

42. The method of claim 41, further comprising:
cleaning or etching the first portion of the metal surgical implant or component before anodically treating the first portion.

43. The method of claim 42, further comprising:
cleaning or etching the second portion of the metal surgical implant or component before passivating the second portion.

44. The method of claim 41, wherein the first portion is a bone integrating portion.

45. The method of claim 41, wherein the step of anodically treating precedes the step of passivating.

46. The method of claim 41, wherein the step of passivating precedes the step of anodically treating.

47. The method of claim 41, wherein the phosphorus-containing compound is selected from the group consisting of phosphoric acid, alkali metal dihydrogen phosphate, alkali metal hydrogen phosphate, and combinations thereof.

48. The method of claim 41, wherein the first portion comprises a substantially nonporous, solid surface.

49. The method of claim 41, wherein the anodically treated first portion is characterized by promoting greater bone tissue growth, greater marrow tissue growth, and lower fibrous tissue growth than an untreated metal surgical implant or component.

50. The method of claim 41, wherein the second portion is passivated in a solution comprising between 20 and 45 volume percent nitric acid.

51. A method of treating a metallic surgical implant, a metallic component for a surgical implant, or a metallic component which is to be formed into a surgical implant, the method comprising:
performing anodic oxidation on a surface of the surgical implant or component, wherein the surface consists at least partly of a metal selected from titanium, titanium alloy, zirconium, zirconium alloy, stainless steel, or a combination thereof, and wherein the anodic oxidation is performed with the surface disposed in an aqueous or nonaqueous solution consisting essentially of a phosphorus-containing compound.

52. The method of claim 51, wherein the phosphorus-containing compound is a phosphate-containing compound.

53. A method of treating a metallic surgical implant, a metallic component for a surgical implant, or a metallic component which is to be formed into a surgical implant, the method comprising:
performing anodic oxidation on a surface of the surgical implant or component, wherein the surface consists at least partly of a metal selected from titanium, titanium alloy, zirconium, zirconium alloy, stainless steel, or a combination thereof, wherein the anodic oxidation is performed with the surface disposed in a solution comprising a phosphorus-containing compound.

54. The method of claim 53, wherein the phosphorus-containing compound is a phosphate-containing compound.

55. A method for improving the biocompatibility of a surgical implant or component for a surgical implant, consisting essentially of:
anodically treating a titanium or titanium alloy surface of the surgical implant or component.

56. A method for improving the biocompatibility of a surgical implant or component for a surgical implant, comprising:
anodically treating a titanium or titanium alloy surface of the surgical implant or component in a phosphorus-containing solution, the surface having no heat treatment above 120 C after the anodic treatment.

57. The method of claim 56, wherein the phosphorus-containing compound is a phosphate-containing compound.

58. A method for improving the biocompatibility of a surgical implant or component for a surgical implant, consisting essentially of:
anodically treating a surface of a surgical implant or component having less than 98 percent titanium, wherein the surface takes on a color selected from purple, gold, and blue.

59. A method for improving the biocompatibility of a surgical implant or component for a surgical implant, comprising:
anodically treating a surface of a metal forming at least a portion of the surgical implant or component at a controlled voltage between 10 and 150 Volts, wherein the surface takes on a color selected from purple, gold, and blue, and wherein the metal is a titanium alloy having less than 98 percent titanium.

60. The method of claim 59, wherein the controlled voltage is a constant voltage.

61. The method of claim 59, wherein the anodic treatment is carried out at constant current, constant electrode potential, constant voltage, or combinations thereof.

62. A method for improving the biocompatibility of a surgical implant or component for a surgical implant, comprising:
performing a single anodic treatment on a metal surface of the surgical implant disposed in a substantially calcium-free solution comprising a phosphorus-containing compound.

63. The method of claim 62, wherein the phosphorus-containing compound is a phosphate-containing compound.

64. A method for improving the biocompatibility of a surgical implant or component for a surgical implant, comprising:
anodically treating a metal surface of the surgical implant or component disposed in an aqueous solution consisting essentially of one or more phosphorus-containing compounds.

65. The method of claim 64, wherein the phosphorus-containing compound is a phosphate-containing compound.

66. The method of claim 65, wherein the phosphate-containing compound is phosphoric acid.

67. The surgical implant formed by any of claims 62.

68. A biocompatible surgical implant, at least a part of the surgical implant comprising:
one or more metal selected from titanium, tantalum, niobium, hafnium, zirconium, and alloys thereof; and
an electrochemically grown anodic oxidation film formed over the one or more metal, wherein the anodic oxidation film comprises the one or more metal, phosphorus atoms and oxygen atoms and does not comprise a calcium phosphate layer.

69. The implant of claim 68, wherein the phosphorus atoms are provided by a component selected from phosphorus, phosphorus oxides, titanium phosphorus oxides and combinations thereof.

70. The implant of claim 68, wherein a portion of the phosphorus atoms are provided by phosphate.

71. The implant of claim 68, wherein the phosphorus atoms have a concentration between about 1 mole % and about 15 mole % at the surface of the substrate.

72. The implant of claim 68, wherein there is no prior electrochemically grown layer of titanium oxide between the metal and the surface comprising phosphorus and oxygen.

73. The implant of claim 68, wherein the one or more metal is Ti-6Al-4V.

74. The implant of claim 68, wherein the one or more metal includes a titanium alloy having an element selected from molybdenum, zirconium, iron, aluminum, vanadium and combinations thereof.

75. The implant of claim 68, wherein the implant is an orthopedic implant.

76. The implant of claim 68, wherein the implant is a dental implant.

77. The implant of claim 68, wherein the implant is an orthopedic fixation device.

78. The implant of claim 68, wherein the implant is a device selected from an orthopedic joint replacement and a prosthetic disc for spinal fixation.

79. The implant of claim 68, wherein the metal comprises:
a solid inner portion; and
a porous outer layer secured to the solid inner portion.

80. The implant of claim 79, wherein the pores of the porous layer are dimensioned so that body tissue can grow into pores in the porous outer layer.

81. The implant of claim 80, wherein the body tissue is selected from bone, marrow and combinations thereof.

82. The implant of claim 79, wherein the porous outer layer is made from the same material as the solid inner portion.

83. The implant of claim 79, wherein the porous outer layer is made from a different material than the solid inner portion.

84. The implant of claim 79, wherein the porous outer layer is made from a material selected from titanium and titanium alloys.

85. The implant of claim 79, wherein the porous outer layer comprises sintered metal particles.

86. The implant of claim 79, further comprising:
a coating of hydroxyapatite deposited on internal surfaces and external surfaces of the porous outer layer without blocking the pores.

87. The implant of claim 86, wherein the hydroxyapatite coating is applied by a method selected from plasma deposition and electrodeposition.

88. The implant of claim 68, wherein the surface incorporates phosphorus to a depth of less than about 1 micron.

89. The implant of claim 88, wherein the surface incorporates phosphorus to a depth between about 0.1 microns to about 0.9 microns.

90. The implant of claim 89, wherein the surface incorporates phosphorus to a depth between about 0.2 microns and about 0.5 microns.

91. The implant of claim 68, wherein the surface incorporates phosphorus to a depth between about 0.2 microns and about 5 microns.

92. The implant of claim 68, wherein the surface incorporates phosphorus to a depth between about 0.5 microns and about 5 microns.

93. The implant of claim 68, wherein the surface incorporates phosphorus to a depth greater than about 1 micron.

94. The implant of claim 68, characterized in that the percentage coverage of the electrochemically treated surface of the biocompatible implant, after being implanted in a dog for six months, is in the range of 20 to 50 % bone, 12 to 22 % marrow, 22 to 44 % fibrous tissue, and 19 to 25 % titanium beads.

95. The implant of claim 68, wherein the metal comprises a solid inner portion and a porous outer layer of metal beads secured to the solid inner portion.

96. The implant of claim 68, wherein the anodically treated metal surface has a phosphorus concentration gradient that increases with increasing thickness of the film.

97. The implant of claim 96, wherein the surface experiences a corrosion rate of less than 10⁻⁸ A/cm² in contact with body fluids.

98. The implant of claim 68, wherein the metal is the alloy Zr_{41.2} Ti_{13.8} Ni₁₀ Cu_{12.5} Be_{22.5}.

99. The implant of claim 68, wherein the anodic oxide film is substantially calcium-free.

100. The implant of claim 68, wherein the anodic oxide film is a barrier film.

101. The implant of claim 68, wherein the oxide film has a thickness of less than 2000 Angstroms.

102. The method of claim 55, wherein the surgical implant or component is disposed in a substantially calcium-free solution of phosphoric acid during the anodic treatment.

103. The method of claim 102, wherein the surface comprises a titanium-containing metal alloy having less than 98 percent titanium.
